(19) United States
(12) Patent Application Publication
(10) Pub. No.: US 2024/0075167 A1
(43) Pub. Date: Mar. 7, 2024

(54) METHOD OF GENE DELIVERY TO RETINAL ASTROCYTES

(71) Applicants: The Board of Trustees of the Leland Stanford Junior University, Stanford, CA (US); The United States Government as Represented by The Department of Veterans Affairs, Washington, DC (US)

(72) Inventors: Jeffrey L. Goldberg, Menlo Park, CA (US); Evan Cameron, Monkton, MD (US)

(21) Appl. No.: 18/271,618

(22) PCT Filed: Feb. 2, 2022
(86) PCT No.: PCT/US2022/014928

§ 371 Date: Jul. 10, 2023

Related U.S. Application Data
(60) Provisional application No. 63/145,171, filed on Feb. 3, 2021.

(51) Int. Cl. A61K 48/00; A61K 9/00; A61P 27/02; C12N 9/22; C12N 15/86

(52) U.S. Cl. CPC A61K 48/0075; A61K 9/0048; A61P 27/02; C12N 9/22; C12N 15/86; C12N 2310/20; C12N 2750/14143

(57) ABSTRACT

The present invention relates to a method for selectively transducing retinal and optic nerve head (ONH) astrocytes using adeno-associated virus serotype 5 (AAV5) in combination with a modified glial fibrillary acidic protein promoter (gfaABC₁D) for delivery of gene therapies (recombinant DNA, shRNA, Crispr/Cas9) to treat glaucoma or other optic neuropathies

### CROSS REFERENCE TO RELATED APPLICATION

The present application claims the benefit of and priority to U.S. Provisional Patent Application No. 63/145,171, filed Feb. 3, 2021, the entire disclosure of which is hereby.

### INCORPORATION BY REFERENCE OF SEQUENCE LISTING PROVIDED AS A TEXT FILE

A Sequence Listing is provided herewith in a text file, (S20-457_STAN-1812WO_Seq_Listing_ST25.txt), created on Feb. 1, 2022, and having a size of 2000 bytes. The contents of the text file are incorporated herein by reference in its entirety.

### BACKGROUND

Glaucoma is the most common cause of irreversible blindness characterized by progressive degeneration and loss of retinal ganglion cells (RGC). Elevated intraocular pressure (IOP) is the only modifiable risk factor for glaucoma; the effects of which manifest at the optic nerve head (ONH) in the form of cupping and neuroretinal thinning. These structural changes are directly reflective of RGC axon degeneration as well as changes in the ONH itself which is composed principally of astrocytes that provide structural and metabolic support to RGC axons as they exit the retina. In addition to directly damaging RGC axons, increased IOP leads to functional changes in ONH astrocytes that contribute to and exacerbate RGC degeneration and death. Clinically, lowering IOP can slow the progression of RGC cell death and prevent vision loss but requires application of daily eye drops which are subject to low adherence, less effective in late-stage disease, and do not directly address the contributions of ONH astrocytes. Thus, methods to specifically deliver therapeutic molecules to ONH astrocytes is critically important to the development of gene therapies for the treatment of glaucoma and other optic neuropathies.

Currently, there are no methods to deliver gene therapies specifically to retinal and optic nerve head astrocytes for the treatment of glaucoma and inner retinal dystrophies.

### SUMMARY

Methods and compositions are provided for in vivo gene delivery to selectively modify retinal astrocytes. Genetic sequences may be delivered to astrocytes for therapeutic purposes, including without limitation for the treatment of glaucoma, inner retinal dystrophies and other optic neuropathies.

A therapeutic nucleic acid composition is provided, comprising a therapeutic nucleic acid sequence comprising a first viral inverted terminal repeat sequence, a promoter, a transgene, a posttranslational regulatory element, a polyadenylation sequence and a second viral inverted terminal repeat sequence. In some embodiments the nucleic acid sequence is encapsulated by a viral capsid. The therapeutic nucleic acid may be provided in a vector, e.g. an AAV packaging plasmid. In some embodiments, expression of the transgene sequence is controlled by a modified glial fibrillary acidic protein (GFAP) promoter, gfaABC₁D or a variant thereof. In some embodiments an effective unit dose of the composition is provided for therapeutic purposes.

In some embodiments the therapeutic composition is administered to an individual for the treatment of glaucoma and inner retinal dystrophies. In some embodiments, the composition is delivered through intravitreal administration. In some embodiment, the composition is administered for the treatment or prophylaxis of conditions that lead to inner retina degeneration including, without limitation, glaucoma, ischemic optic neuropathy, diabetic retinopathy, etc.

In one embodiment, expression of the transgene sequence is controlled by the modified GFAP promoter, gfaABC₁D, or a variant thereof, to enable specific expression in retinal and ONH astrocytes. In some embodiments, the transgene is expressed specifically in retinal and ONH astrocytes and not in microglia, ganglion cells (RGCs) or photoreceptors (PRCs). In other embodiments a different astrocyte specific promoter may be used, e.g. a human gaf2 promoter, a human ALDH1 L1 promoter, a human EAAT₁ promoter, etc.

In some embodiments, the transgene within the therapeutic nucleic acid sequence encodes one or more elements of a programmable gene editing system, e.g. a CRISPR/Cas9 system. In other embodiments, the transgene may comprise, but not be limited to, recombinant DNA, shRNA, miRNA, etc.

In some embodiments, the AAV vector comprises AAV2 inverted terminal repeats (ITRs). In some embodiments, other AAV ITRs may be used which include but are not limited to AAV1, AAV2, AAV3, AAV4, AAV6, AAV7, AAV8, AAV9, etc.

In a preferred embodiment, the nucleic acid composition is packaged in ultra-purified viral particle. In some embodiments, the viral capsid (or particle) is an AAV5 viral capsid. Ultra-purified refers to the level of contamination present within a composition. In some embodiments, ultra-purified means that the level of contamination is at least <0.01% of the total solution. In some embodiments, ultra-purified refers to a level of contamination that is 0.01-0.005%, 0.005-0.001%, 0.001-0.0005%, 0.0005-0.0001%, or <0.0001% of the total composition. In some embodiments, other AAV viral capsids may be used which include but are not limited to AAV1, AAV2, AAV3, AAV4, AAV6, AAV7, AAV8, AAV9, etc.

In some embodiments, an effective dose for efficient optic nerve head astrocyte genetic modification comprises at least >10⁸ viral particles/mL. In some embodiments, an effective dose comprises 10⁸-10⁹ viral particles/mL, 10⁹-10¹⁰ viral particles/mL, 10¹⁰-10¹¹ viral particles/mL, 10¹¹-10¹² viral particles/mL, 10¹²-10¹³ viral particles/mL or >10¹³ viral particles/mL.

In a preferred embodiment, the gene therapy is packaged in a single stranded, double stranded, or self-complementary AAV vector.

### BRIEF DESCRIPTION OF DRAWINGS

FIG. 1: Modified glial fibrillary acid protein (GFAP) promoter sequence, gfaABC₁D, promotes gene expression in retinal astrocytes, SEQ ID NO:1. gfaABC₁D is a truncated human GFAP promoter consisting of regulatory elements that enable astrocyte specific gene expression at levels higher than the full-length GFAP promoter. These features combined with its smaller size make gfaABC₁D an ideal candidate to mediate adeno-associated virus (AAV) transgene expression in injured (a.k.a. reactive) astrocytes for the treatment of neurological disease. Notably, gfaABC₁D was previously found to promote astrocyte specific gene expression in the multiple regions in the brain and spinal cord including the brain stem, hippocampus, cortex, and olfactory bulbs, but its ability to promote expression in the visual system has not been reported.

FIG. 2: Generation of AAV5.gfaABC₁D viral vectors for in vivo transduction of optic nerve head (ONH) astrocytes. In the present invention, targeted transgene expression in ONH astrocytes is endowed through the combination of two critical elements: A. an AAV packaging vector including the gfaABC₁D promoter and B. an AAV5 viral capsid. The packaging vector includes the gfaABC₁D promoter upstream of a transgene coding sequence (which could be recombinant DNA, shRNA, Crisper/Cas9, etc.) followed by a woodchuck posttranscriptional regulatory element (WPRE), and a bovine growth hormone polyadenylation sequence (bGH poly(A)). WPRE increases viral gene expression in packaging cells necessary for production of the high titers required for this invention, and a bGH poly (A) ensures faithful, robust transgene expression in transduced cells. These elements are flanked by two AAV2 inverted terminal repeats (ITR) required for AAV viral packaging. AAV5.gfaABC₁D viral particles should be generated using the herein described packaging vector following standard AAV viral production protocols with an AAV5 rep-cap, and helper plasmids. The virus should be concentrated by polyethylene glycol (PEG) precipitation and further concentrated and purified by cesium chloride (CsCl) gradient ultracentrifugation to produce in vivo grade AAV5.gfaABC₁D virus at titers necessary to efficiently transduce ONH astrocytes (21×10¹² viral particle/mL).

FIG. 3: A method to specifically transduce astrocytes in the mouse retina. A. Intravitreal injection of 2 μls of 1×10⁹ vps/mL of AAV5.gfaABC₁D transduces optic nerve head astrocytes in mice. B. Virus injection may be accomplished using a preloaded Hamilton syringe inserted at a 60° angle ˜1 mm into the vitreous cavity just below the limbus, as commonly understood by one of ordinary skill in the art. C-D. Retinal flat-mount showing AAV5.gfaABC₁D-tdTomato preferentially transduces optic nerve head astrocytes (dashed box in C, magnified in D) and a small subset of nerve fiber layer (NFL, solid arrow) astrocytes identified by GFAP staining (dotted box in C, magnified in D) but not retinal ganglion cells (RGCs) stained with RNA-binding protein with multiple splice (RBPMS). RGC transduction (tdTomato+/RBPMS+) from flat mounts quantified in FIG. 6. Scale in C is 500 μm. Scale in D is 20 μm.

FIG. 4: Intravitreal injection of AAV5.gfaABC₁D preferentially transduces optic nerve head (ONH) astrocytes in mice. A. Cross section of an AAV5.gfaABC₁D-tdTomato injected retina stained with GFAP, Sox9, and DAPI showing high tdTomato expression in the ONH region. Scale is 200 μm. B. ONH region (magnified from dashed box from A) defined vertically by the area between the glial lamina and the surface of the retina and horizontally from the center of the optic disc 200 μm to the optic disc rim. Scale is 50 μm. C. Central retina (magnified from dotted box from A) showing a small subset of nerve fiber layer (NFL, solid arrow) astrocytes as well as Mueller glial (MG, open arrow) are transduced by AAV5.gfaABC₁D-tdTomato. Scale is 50 μm. D. GFAP and Sox9 staining showing high astrocyte transduction (solid arrows) in the defined ONH region between the glial lamina and optic disc rim (magnified from B). E. NFL astrocytes (solid arrows) and Muller glia (MG) in the inner nuclear layer (INL) (open arrow) showing low AAV5. gfaABC₁D-tdTomato expression (magnified from C). Photoreceptors in the outer nuclear layer (ONL) are not transduced (C and E). Scale is 25 μm. Quantification of AAV5.gfaABC₁D.tdTomato transduction efficiencies shown in FIG. 6.

FIG. 5. AAV5-gfaABC₁D does not transduce microglia in the mouse retina. A. Representative images of IBA1+ stained optic nerve heads showing microglia are not transduced following intravitreal injection of AAV5.gfaABC₁D-tdTomato. Scale is 100 μm. B. Magnified ONH region (dashed box from A) shows IBA+ cells (green; open arrows) do not colocalize with tdTomato (solid arrows; red). Scale is 20 μm. IBA1+ cell transduction (tdTomato+/IBA1+) quantified in FIG. 6.

FIG. 6. Quantification of AAV5.gfaABC₁D-tdTomato transduction in the mouse retina. Intravitreal injection of 2 μls of 1×10^{\hat{ }9} vps/mL of AAV5.gfaABC₁D preferentially transduces optic nerve head (ONH) astrocytes (˜95%), a small subset of nerve fiber (NFL) astrocytes (˜7.5%), and less than 2.5% of muller glia (MG). AAV5.gfaABC₁D does not transduce retinal ganglion cells (RGCs), photoreceptors (PRCs), microglial (IBA+), or any other cell type in the retina. RGC transduction quantified in flat mounts retinas stained with RBPMS (FIG. 3C-D). ONH and NFL astrocytes defined by GFAP and Sox9 expression in the ONH region and retina, respectively, Muller glia (MG) defined by Sox9 and DAPI in the inner nuclear layer (INL), photoreceptors defined by DAPI in the outer nuclear layer (ONL) (FIG. 4D-E), and microglia defined by IBA1 expression (FIG. 5) and quantified in retinal cross sections. One-way ANOVA; ** p<0.01.

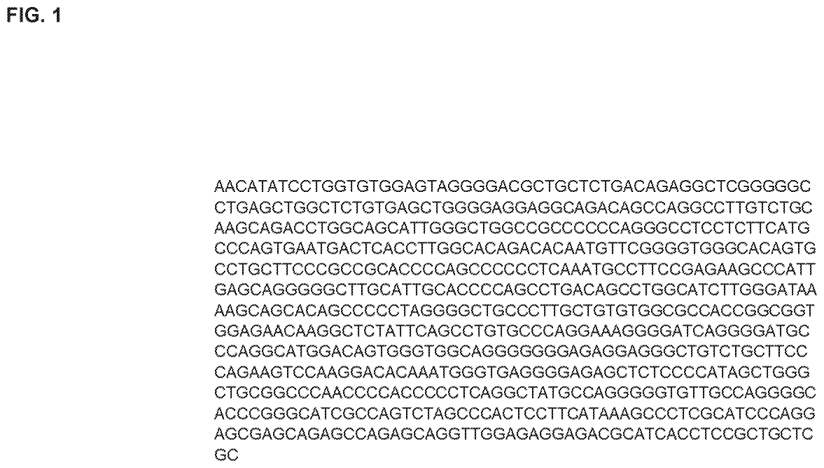

### DETAILED DESCRIPTION

### Definitions

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction in bacterial titers).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; avians, and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “therapeutically effective amount” or “efficacious amount” means the amount of a compound that, when administered to a mammal or other subject for treating a disease, condition, or disorder, is sufficient to affect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical formulation that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical formulation” is meant to encompass a formulation suitable for administration to a subject, such as a mammal, especially a human. In general a “pharmaceutical formulation” is sterile, and preferably free of contaminants that are capable of eliciting an undesirable response within the subject (e.g., the compound(s) in the pharmaceutical formulation is pharmaceutical grade). Pharmaceutical formulations can be designed for administration to subjects or patients in need thereof via a number of different routes of administration including for the purposes of the disclosed methods, intravitreal, topically applied to the eye, and the like.

Promoters and Vectors

Aspects of the present invention encompass expression cassettes and/or vectors comprising polynucleotide sequences of interest for expression in targeted cells. The polynucleotides can comprise promoters operably linked to the region of the polynucleotide that encodes e.g., a CRISPR/Cas system, a therapeutic transgene, an RNAi sequence, and the like. Targeted expression is accomplished using a cell-selective or cell-specific promoter. Examples are promoters for somatostatin, parvalbumin, GABAa6, L7, and calbindin. Other cell specific promoters can be promoters for kinases such as PKC, PKA, and CaMKII; promoters for other ligand receptors such as NMDAR1, NNIDAR2B, GluR2; promoters for ion channels including calcium channels, potassium channels, chloride channels, and sodium channels; and promoters for other markers that label classical mature and dividing cell types, such as calretinin, nestin, and beta3-tubulin.

Specifically, where expression of a subject polynucleotide in an optic nerve head astrocyte is desired, a promoter of interest with astrocyte selective expression is used. Promoters of interest include but are not limited to, full length human gaf2 promoter, full length gfa28 promoter, truncated human gaf2 promoter gfaABC₁D, full length human ALDH1L1 promoter, full length human EAAT1 promoter, etc. In some embodiments the promoter is gfaABC₁D, SEQ ID NO:1.

Variants of the above discussed promoters may also be used. In some instances, a suitable variant comprises a nucleotide sequence having 30% or more, 40% or more, 50% or more, 60% or more, 70% or more, 75% or more, 80% or more, 85% or more, 90% or more, 95% or more, 99% or more or 100% nucleotide sequence identity to their corresponding “reference” promoter, e.g. SEQ ID NO:1. A person of skill in the art will recognize that various promoters drive expression in various cell types, and will be able to decide on which promoter to use for their desired outcome.

Glial fibrillary acidic protein (GFAP) is the major intermediate filament protein in astrocytes, one of the most abundant cell types in the vertebrate central nervous system (CNS) (Bignami et al., 1972; Eng et al., 1971). Its expression is used as a marker of astrocyte differentiation (Bonni et al., 1997), and its upregulation is the hallmark of gliosis in CNS injury (Eng and Ghirnikar, 1994; Ransom et al., 2003). Therefore, the regulatory elements of the GFAP gene have been studied to understand both developmental signaling for astrocyte maturation and the response of astrocytes to CNS injury (Bonni et al., 1997; Kahn et al., 1997; Martin et al., 2003). In addition, knowledge gained from the identification of GFAP regulatory elements has been used to develop promoters capable of targeting transgene expression to astrocytes (Brenner et al., 1994; de Leeuw et al., 2006). For example, the gfa2 promoter, which spans bp 22163 to 147 of the human GFAP gene, has been widely used for this purpose (reviewed in Su et al., 2004).

In a preferred embodiment, the truncated gfa2 promoter gfaABC₁D (provided herein as SEQ ID NO:1) or a variant thereof is used. The GfaABC₁D promoter is a compact GFAP promoter with the size of 681 bp. It was derived from the conventional 2.2 kb human GFAP promoter by deleting 5′ nucleotides −2163 to −1757 and an internal segment from −1257 to −132. gfaABC₁D previously displayed expression properties in transgenic mice indistinguishable from the 2.2 kb promoter. This promoter allows for greater flexibility in creating therapeutic AAV constructs with less transgene size restrictions, which can be a drawback of using AAV for gene delivery. In some embodiments a variant of the gfaABC₁D may be used. A suitable gfaABC₁D promoter variant comprises a nucleotide sequence having 30% or more, 40% or more, 50% or more, 60% or more, 70% or more, 75% or more, 80% or more, 85% or more, 90% or more, 95% or more, 99% or more or 100% nucleotide sequence identity to the gfaABC₁D promoter of SEQ ID NO:1.

Utilizing a viral vehicle to deliver genetic material into cells allows direct targeting of pathogenic molecules and restoration of function. The retina is an advantageous target for gene therapy due to its easy access, confined non-systemic localization, partial immune privilege, and well-established definitive functional readouts. Because AAV is non-pathogenic and cannot reproduce itself without helper viruses, it has served as a primary vehicle for gene therapy. It is a single-stranded DNA virus that stably and efficiently infects a wide variety of cells in multiple tissues. AAV-mediated gene therapies specifically targeted to astrocytes within the ONH are disclosed herein.

In some embodiments, the vector is a recombinant adeno-associated virus (AAV) vector. AAV vectors are DNA viruses of relatively small size that can integrate, in a stable and site specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV could infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87: 2211-2215; Samulski et al, EMBO J., 1991. 10: 3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134: 52-63), while AAV1-4 and AAV6 are all found in the study of adenovirus (Ursula Bantel-Schaal, Hajo Delius and Harald zur Hausen. J. Viral., 1999. 73: 939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. ME Bloom, RMLinden, C RParrish, Eds.) p 5-14, Rudder Arnold, London, U K (2006); and D E Bowles, J E Rabinowitz, R J Samulski “The Genus Dependovirus” (J R Kerr, SF Cotmore. ME Bloom, R M Linden, C R Parrish, Eds.) p 15-23, Rudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCTIUS2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAV1, AAV2, AAV3, AAV4, AAV5, AAV6, AAV7, AAV8, AAV9, AAVIO, AAVII, AAV12, AAV13, AAV14, AAV15, and AAV16). Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

Transqenes

The methods described herein are useful in the delivery of therapeutic transgenes to retinal astrocytes. In some embodiments, the transgene encodes, without limitation, a programmable gene editing system, e.g. a CRISPR/Cas9 system, a coding sequence for a protein of interest, shRNA, miRNA, and the like.

A transgene of interest (i.e., a gene that encodes a given biologically active molecule) can be inserted into a vector using standard techniques known in the art. The nucleic acid and amino acid sequences of the human (and other mammalian) genes encoding suitable biologically active molecules are known.

In some embodiments, the transgene encodes a neurotrophic factor. In some embodiments, a neurotrophic factor is selected from the group consisting of: brain derived neurotrophic factor (BDNF), nerve growth factor (NGF), neurotrophin-3, ciliary neurotrophic factor (CNTF), an ephrin, glial cell line-derived neurotrophic factors (GDNF) and a combination thereof. In some embodiments, the therapeutic agent comprises an optogenetic actuator. In some embodiments, the optogenetic actuator is selected from a bacteriorhodopsin, a halorhodopsin, a channelrhodopsin, a microbial sensory rhodopsin, a mammalian rhodopsin, a cone opsin, a melanopsin, or a combination thereof.

For example, ciliary neurotrophic factor (CNTF) is a protein that is involved in promoting neurotransmitter synthesis and neurite outgrowth in neuronal populations. CNTF is a survival factor for neuronal cells, including neurons and oligodendrocytes, and has been demonstrated to have a protective role for photoreceptors.

Vascular endothelial growth factor (VEGF) is a signaling protein involved in both vasculogenesis, the formation of the embryonic circulatory system, and angiogenesis, the growth of blood vessels from pre-existing vasculature. While VEGF is mostly known for its effects on cells of the vascular endothelium, it also affects a broad range of other cells types, e.g., stimulation monocyte/macrophage migration, neurons, cancer cells, kidney epithelial cells, etc. There are a number of proteins within the VEGF family, which arise as a result of alternate splicing of mRNA. The various splice variants impact the function of VEGF, as they determine whether the resulting proteins are pro- or anti-angiogenic. Additionally, the splice variants also effect the interaction of VEGF with heparin sulfate proteoglycans (HSPGs) and neuripilin co-receptors on the cell surface, which, in turn, enhances the ability of VEGF to bind to and activate VEGF signaling receptors (VEGFRs). Structurally, VEGF belongs to the PDGF family of cysteine-knot growth factors, and, thus, several closely-related proteins exist, i.e., placenta growth factor (P1GF), VEGF-B, VEGF-C and VEGF-D, which together comprise the VEGF sub-family of growth factors. VEGF itself is commonly referred to as VEGF-A in order to differentiate it from these other, related growth factors.

Platelet Derived Growth Factor (PDGF) is a growth factor that also plays a role in angiogenesis. Multiple forms of PDGF exists, composed dimers containing two A chains (AA), two B chains (BB), or a mixed AB chain (AB). PDGF is a potent mitogen for pericytes, a class of cells that serve as support for endothelial cell growth. PDGF receptor (PDGFR) exists in two forms, alpha and beta. PDGFR beta has the highest affinity for PDGF-BB and has been shown to exert anti-angiogenic biological effect as a secreted protein in either fusion protein—Fc form or as an extracellular soluble receptor. Recently, potent synergistic anti-angiogenic activity has been demonstrated in mouse ocular vascular neogenesis models involving the combination of anti-VEGF molecules and antagonistic PDGF molecules. Thus a combination anti-PDGF, anti-VEGF therapy may exert a higher anti-angiogenic activity than anti-VEGF therapy alone.

Other examples of proteins, including cytokines and other immune or glial modulators of interest, include, but are not limited to, BDNF, TGF-β, GDNF, NGF, bFGF, aFGF, IL-1p, IL-10, IFN-β, and IFN-α. See, for example, BDNF (Lipsky and Marini, 2007, Ann NY Acad Sci, 1122:130-43), TGF-β(see Krieglstein et al J. Physiol Paris, 2002, 96(1-2):25-30), GDNF (see Suzuki et al. PLoS, 2007, 2(8):e689), NGF (Chun and Patterson, J C B, 1977 (75): 694-704), bFGF (see Meijs et al. J Neurotrauma 2004, 21(10):1415-30), aFGF (Lipton et al. 1988, PNAS 85: 2388-2392), IL-10 (Boyd et al., Invest. Ophthalmol. Vis. Sci. 44:5206-5211), IFN-(3 (Sattler et al., Exp Neurol, 2006, 201(1): 172-81), and IFN-α (He Yang et al. PNAS, 2000, 97(25): 13631-13636).

In other embodiments a transgene encodes shRNA or an RNAi sequence. RNAi is a phenomenon in which the introduction of dsRNA into a diverse range of organisms and cell types causes degradation of the complementary mRNA. In the cell, long dsRNAs are cleaved into short (e.g., 21-25 nucleotide) small interfering RNAs (siRNAs), by a ribonuclease. The siRNAs subsequently assemble with protein components into an RNA-induced silencing complex (RISC), unwinding in the process. The activated RISC then binds to complementary transcripts by base pairing interactions between the siRNA antisense strand and the mRNA. The bound mRNA is then cleaved and sequence specific degradation of mRNA results in gene silencing. As used herein, “silencing” refers to a mechanism by which cells shut down large sections of chromosomal DNA resulting in suppressing the expression of a particular gene. Without being bound by theory, the RNAi machinery appears to have evolved to protect the genome from endogenous transposable elements and from viral infections. Thus, RNAi can be induced by introducing nucleic acid molecules complementary to the target mRNA to be degraded, as described herein.

In some embodiments, a viral particle of the present disclosure comprises a class 2 CRISPR/Cas effector protein (or a nucleic encoding the protein), e.g., as an endonuclease. In class 2 CRISPR systems, the functions of the effector complex (e.g., the cleavage of target DNA) are carried out by a single protein (which can be referred to as a CRISPR/Cas effector protein)—where the natural protein is an endonuclease. As such, the term “class 2 CRISPR/Cas protein” or “CRISPR/Cas effector protein” is used herein to encompass the effector protein from class 2 CRISPR systems—for example, type II CRISPR/Cas proteins (e.g., Cas9), type V CRISPR/Cas proteins (e.g., Cpf1/Cas12a, C2c1/Cas12b, C2C3/Cas12c), and type VI CRISPR/Cas proteins (e.g., C2c2/Cas13a, C2C7/Cas13c, C2c6/Cas13b). Class 2 CRISPR/Cas effector proteins include type II, type V, and type VI CRISPR/Cas proteins, but the term is also meant to encompass any class 2 CRISPR/Cas protein suitable for binding to a corresponding guide RNA and forming a ribonucleoprotein (RNP) complex.

In some embodiments, an RNA-guided endonuclease is a fusion protein that is fused to a heterologous polypeptide (also referred to as a “fusion partner”). In some cases, an RNA-guided endonuclease is fused to an amino acid sequence (a fusion partner) that provides for subcellular localization, i.e., the fusion partner is a subcellular localization sequence (e.g., one or more nuclear localization signals (NLSs) for targeting to the nucleus, two or more NLSs, three or more NLSs, etc.). In some embodiments, an RNA-guided endonuclease is fused to an amino acid sequence (a fusion partner) that provides a tag (i.e., the fusion partner is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6XHis tag; a hemagglutinin (HA) tag; a FLAG tag; a Myc tag; and the like). In some cases, the fusion partner can provide for increased or decreased stability (i.e., the fusion partner can be a stability control peptide, e.g., a degron, which in some cases is controllable (e.g., a temperature sensitive or drug controllable degron sequence).

A nucleic acid that binds to a class 2 CRISPR/Cas effector protein (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence (also referred to herein as a targeting sequence), which is a nucleotide sequence that is complementary to a sequence of a target nucleic acid. In some embodiments, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some embodiments, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

In some cases, a nucleic acid payload includes or encodes a gene editing tool (i.e., a component of a gene editing system, e.g., a site specific gene editing system such as a programmable gene editing system). For example, a nucleic acid payload can include one or more of: (i) a CRISPR/Cas guide RNA, (ii) a DNA encoding a CRISPR/Cas guide RNA, (iii) a DNA and/or RNA encoding a programmable gene editing protein such as a zinc finger protein (ZFP) (e.g., a zinc finger nuclease—ZFN), a transcription activator-like effector (TALE) protein (e.g., fused to a nuclease—TALEN), a DNA-guided polypeptide such as Natronobacterium gregoryi Argonaute (NgAgo), and/or a CRISPR/Cas RNA-guided polypeptide (e.g., Cas9, CasX, CasY, Cpf1, and the like); (iv) a DNA donor template; (v) a nucleic acid molecule (DNA, RNA) encoding a site-specific recombinase (e.g., Cre recombinase, Dre recombinase, Flp recombinase, KD recombinase, B2 recombinase, B3 recombinase, R recombinase, Hin recombinase, Tre recombinase, PhiC31 integrase, Bxb1 integrase, R4 integrase, lambda integrase, HK022 integrase, HP1 integrase, and the like); (vi) a DNA encoding a resolvase and/or invertase (e.g., Gin, Hin, yδ3, Tn3, Sin, Beta, and the like); and (vii) a transposon and/or a DNA derived from a transposon (e.g., bacterial transposons such as Tn3, Tn5, Tn7, Tn9, Tn10, Tn903, Tn1681, and the like; eukaryotic transposons such as Tc1/mariner super family transposons, PiggyBac superfamily transposons, hAT superfamily transposons, PiggyBac, Sleeping Beauty, Frog Prince, Minos, Himar1, and the like). In some cases a subject delivery molecule is used to deliver a protein payload, e.g., a gene editing protein such as a ZFP (e.g., ZFN), a TALE (e.g., TALEN), a CRISPR/Cas RNA-guided polypeptide (e.g., Cas9, CasX, CasY, Cpf1, and the like), a site-specific recombinase (e.g., Cre recombinase, Dre recombinase, Flp recombinase, KD recombinase, B2 recombinase, B3 recombinase, R recombinase, Hin recombinase, Tre recombinase, PhiC31 integrase, Bxb1 integrase, R4 integrase, lambda integrase, HK022 integrase, HP1 integrase, and the like), a resolvase/invertase (e.g., Gin, Hin, yδ3, Tn3, Sin, Beta, and the like); and/or a transposase (e.g., a transposase related to transposons such as bacterial transposons such as Tn3, Tn5, Tn7, Tn9, Tn10, Tn903, Tn1681, and the like; or eukaryotic transposons such as Tc1/mariner super family transposons, PiggyBac superfamily transposons, hAT superfamily transposons, PiggyBac, Sleeping Beauty, Frog Prince, Minos, Himar1, and the like). In some cases, the delivery molecule is used to deliver a nucleic acid payload and a protein payload, and in some such cases the payload includes a ribonucleoprotein complex (RNP).

In some embodiments, a subject delivery molecule is used to deliver a gene editing tool. In other words in some cases the payload includes one or more gene editing tools. The term “gene editing tool” is used herein to refer to one or more components of a gene editing system. Thus, in some cases the payload includes a gene editing system and in some cases the payload includes one or more components of a gene editing system (i.e., one or more gene editing tools). For example, a target cell might already include one of the components of a gene editing system and the user need only add the remaining components. In such a case the payload of a subject delivery molecule does not necessarily include all of the components of a given gene editing system. As such, in some cases a payload includes one or more gene editing tools.

As would be understood by one of ordinary skill in the art, a gene editing system need not be a system that ‘edits’ a nucleic acid. For example, it is well recognized that a gene editing system can be used to modify target nucleic acids (e.g., DNA and/or RNA) in a variety of ways without creating a double strand break (DSB) in the target DNA. For example, in some cases a double stranded target DNA is nicked (one strand is cleaved), and in some cases (e.g., in some cases where the gene editing protein is devoid of nuclease activity, e.g., a CRISPR/Cas RNA-guided polypeptide may harbor mutations in the catalytic nuclease domains), the target nucleic acid is not cleaved at all. For example, in some cases a CRISPR/Cas protein (e.g., Cas9, CasX, CasY, Cpf1) with or without nuclease activity, is fused to a heterologous protein domain. The heterologous protein domain can provide an activity to the fusion protein such as (i) a DNA-modifying activity (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity), (ii) a transcription modulation activity (e.g., fusion to a transcriptional repressor or activator), or (iii) an activity that modifies a protein (e.g., a histone) that is associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). As such, a gene editing system can be used in applications that modify a target nucleic acid in way that do not cleave the target nucleic acid, and can also be used in applications that modulate transcription from a target DNA.

In some embodiments, gene editing tools are directed to the editing or inhibition of specific genes that have been shown to have detrimental roles in glaucoma or other optic neuropathies. Genes that have detrimental roles in glaucoma or other optic neuropathies include, without limitation, tumor necrosis factor alpha (TNFα), tumor necrosis factor receptor 2 (TNFR2), endothelin-1 (EN-1), Nitric oxide synthase-2 (NOS-2), CD11 b/CD18, Epidermal Growth Factor Receptor (EGFR), matrix metalloproteinases, etc.

Tumor necrosis factor-α (TNF-α) is synthesized primarily by activated monocytes as a 26 kDa precursor that is cleaved proteolytically and secreted as a 17 kDa protein. TNF-α acts via either the low-affinity TNF receptor (TNFR1) or high-affinity TNF receptor (TNFR2). TNF-α is upregulated in optic nerve microglia and astrocytes of glaucoma patients. TNF-α is toxic to immunopurified RGCs and to RGCs in mixed cultures when glia are stressed, although not under resting conditions. Microglia and astrocytes interact in response to acute injury to the optic nerve and in chronic neurodegenerative disease to produce a neurotoxic reactive astrocyte phenotype. This response by astrocytes is largely mediated by the microglial release of interleukin-1 alpha (IL-1α), tumor necrosis factor alpha (TNF-α), and the classical complement component C1q. Altogether, these three factors are necessary and sufficient to induce neuroinflammatory reactive astrocytes, which can then release of a toxin to specifically kill neurons and mature oligodendrocytes. Blocking the activation of these astrocytes (while maintaining an otherwise normal microglia response) using either the neutralizing antibodies to IL-1α, TNF-α, and C1q or an II1a^{-/-}Tnf^{-/-}C1qa^{-/-} triple knockout (tKO) mouse line preserves RGC viability following axotomy.

Intravitreal administration of TNF-α to otherwise normal animals mimicked the degenerative effects of increased IOP, and, conversely, the effects of IOP were eliminated in mice with the TNF-α gene deleted or by immune depletion of TNF-α in wild-type mice. The effect of TNF-α on oligodendrocyte loss was mediated primarily via the TNFR2 receptor, because the cytotoxic effects of either increased IOP or direct administration of TNF-α were eliminated in mice with the TNFR2 gene, although not the TNFR1 gene, deleted. The cytotoxic effect of OH-induced TNF-α may be mediated by CD11 b⁺ microglia, although direct effects on other cell types cannot be ruled out. These cells increased rapidly in numbers and activation after TNF-α administration, and deletion of the gene for integrin β2 (CD11 b/CD18), which is important for microglial activation, prevented the loss of oligodendrocytes and RGCs after increasing IOP or after administering TNF-α.

EN-1: In glaucoma patients plasma concentration of endothelin-1(EN-1) is increased. Endothelin not only further reduces optic nerve head blood flow and impairs anterograde and retrograde axoplasmatic transport, but also activates astrocytes. This is further supported by the fact that patients with vascular dysregulation more often have activated retinal astrocytes, which can be visualized clinically. The effect of endothelin can be partially blocked by a number of different drugs such as calcium channel blockers (CCBs) including magnesium (a physiological CCB), dipyrimadole, or endothelin blockers.

NOS-2: Nitric oxide (NO), also known as the endothelium-derived relaxing factor, is biosynthesized from arginine and oxygen by various nitric oxide synthase (NOS) enzymes. There are three basic forms of NOS: neuronal nitric oxide synthase (nNOS or NOS-1), inducible nitric oxide synthase (iNOS or NOS-2), and endothelial nitric oxide synthase (eNOS or NOS-3). NOS-2 leads to a marked production of nitric oxide. NOS-2 can be inhibited by the drug aminoguanidine, a nucleophilic hydrazine compound. Aminoguanidine is an oral insulin stimulant for type 2 diabetes mellitus. It further seems to prevent the formation of advanced glycation end products. In addition, it is a relative specific inhibitor of NOS2, which is why it was studied in experimental glaucoma. In experimental rat glaucoma models aminoguanidine was capable of preventing the development of glaucoma.

EGFR: Mechanical stress leads to stimulation of EGFR, which, in turn, leads to activation of astrocytes and thereby to an upregulation of NOS-2. Blockage of EGFR, by a tyrosine kinase inhibitor prevents the activation of astrocytes. Treatment not only inhibits the activation of astrocytes but also leads to a reduction of loss of retinal ganglion cells. This indirectly indicates that the activation of astrocytes is relevant in glaucoma.

Matrix metalloproteinases (MMPs): Under normal conditions, astrocytes and microglia express MMP-2 (gelatinase A) in the foot processes near blood vessels. Upon astrocyte and microglial activation, MMP-2 is, however, increased, thereby causing increased permeability of the blood-retinal barrier, angiogenesis, and glial scar formation. Activated microglia also express MMP-3 (stromelysin-1), which in turn activates proMMP-9. MMP-9 has been found to be elevated when there is an increase in the blood-retinal barrier permeability. In line with this, an MMP-9 increase has been shown in diabetic rat retinas when glucose levels rise. MMP-9 has also been implicated in myelin basic protein degradation, and it has therefore been suggested that MMP-9 is associated with demyelination and axonal injury. Finally, both the effects of MMP-9 and MMP-3 have been shown to be enhanced by TNF-α and IL-1, further indicating complex interactions between molecules secreted by different retinal glia.

Methods of Use

As used herein, the term “administration” refers to the administration of a composition (i.e. a composition comprising a nucleic acid sequence) to a subject or system. Administration to an animal subject (e.g., to a human) may be by any appropriate route. For example, in some embodiments, administration may be intradermal, intramedullary, intramuscular, intranasal, intrathecal, intravenous, intraventricular, within a specific organ, mucosal, nasal, oral, subcutaneous, sublingual, topical, tracheal (including by intratracheal instillation), intraocular, vitreal and subconjunctivally. In some embodiments, administration may involve intermittent dosing. In some embodiments, administration may involve continuous dosing (e.g., perfusion) for at least a selected period of time.

In some embodiments, the claimed invention is used to deliver genetic sequences to retinal astrocytes for the treatment of optic neuropathies. In some embodiments, the genetic sequences are expressed in retinal astrocytes and not in microglia, ganglion cells (RGCs) or photoreceptors (PRCs). Optic neuropathies of interest include, but are not limited to, pre-glaucoma., glaucoma, ischemic optic neuropathy (e.g., arteritic or non-arteritic anterior ischemic neuropathy and posterior ischemic optic neuropathy), compressive optic neuropathy, infiltrative optic neuropathy, traumatic optic neuropathy, mitochondrial optic neuropathy (e.g., Leber's optic neuropathy), nutritional optic neuropathy, toxic optic neuropathy, and hereditary optic neuropathy (e.g., Leber's optic neuropathy, Dominant Optic Atrophy, Behr's syndrome). In some embodiments, the glaucoma is primary open-angle glaucoma, angle-closure glaucoma, normal-tension glaucoma, congenital glaucoma, neovascular glaucoma, steroid-induced glaucoma, or glaucoma related to ocular trauma.

Non-limiting examples of symptoms associated with the ocular conditions include the loss of retinal ganglion cell viability in the retina of the eye, glaucoma, optic nerve injury, optic neuritis, optic neuropathies, diabetic retinopathy, central retinal artery occlusion, and central retinal vein occlusion.

Glaucoma is a progressive neuropathy that induces dysfunction and degeneration of the optic nerve and retinal neurons. Of the retinal neurons, retinal ganglion cells (RGCs), which are an essential neuronal subtype that transmit visual information to the brain are the most sensitive to glaucoma. Glaucoma can be triggered when the fluid, called “aqueous humor”, builds up in the front part of the eye. Excess production or reduced draining of the aqueous humor increases the intraocular pressure (IOP), which irreversibly damages the optic nerve and RGCs.

There are two major classes of glaucoma: primary and secondary glaucoma. Primary glaucoma has no detectable cause of the disease. Secondary glaucoma is caused by other medical conditions. Primary glaucoma is classified into open-angle glaucoma (POAG), normal-tension glaucoma (NTG), angle-closure glaucoma and congenital glaucoma. Secondary glaucoma is classified into neovascular glaucoma, pigmentary glaucoma, exfoliation glaucoma and uveitic glaucoma. POAG is the most common type in the United States and is usually associated with an elevated IOP.

One of the characteristic features of glaucoma is structural changes in the optic nerve head (ONH). The optic disc (or simply “disc”) in the ONH is the point where RGC axons gather and exit the eye. The gathered RGC axons form the optic nerve. Under the ophthalmoscopy, the disc can be visualized as blight circle in the center of the eye. In the center of the disc, there is brighter spot, termed as “(optic) cup”. Because a size of cup becomes larger in glaucoma patients, the cup-to-disc ratio (often referred to as CDR) is used to assess disease progression. This change is observed in hypertensive glaucoma (POAG) and in NTG. The structural changes of the tissue cause mechanical deformation of the RGC axons running through this region. The ONH is the most vulnerable region and shows the earliest pathological changes.

Optic nerve atrophy precedes the onset of visual field loss in glaucoma. RGC axons exit the eye through a hole in the sclera that is filled with a mesh-like structure called the lamina cribrosa (LC). Structural change of LC induces ONH cupping. In humans, the LC is enriched in collagens. Because such collagen-rich structures are not observed in rodents, which are often used as glaucoma models, it has been considered that rodents have no LC or ONH cupping. Accumulating evidence has shown that astrocytes-formed LC-like structure can be seen in rodents. The human LC is formed by astrocytes highly expressing glial fibrillary acidic protein (GFAP). The astrocyte-formed mesh-like structure around the optic nerve in rodents is termed glial lamina. Importantly, the axons in the LC are unmyelinated and tightly surrounded by astrocytes. Therefore, changes in astrocytic functions would directly affect the optic nerve.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

Intravitreal injection of AAV5.gfaABC₁D. Mice were anesthetized in accordance with Institutional Animal Care and Use Committee (IACUC)-approved protocols and country-specific regulations. For the purpose of intravitreal injections, isoflurane was administered using a tabletop induction box and nose cone. While animals were under anesthesia, their eyes were kept moist at all times by applying Refresh Tears® lubricating eye drops as needed (one drop per eye every 5 min is sufficient). To prevent the cornea from drying out during intravitreal injections and ONC surgeries liberally apply Refresh Tears® lubricating eye drops as required.

Once anesthetized, the mouse was transferred to a surgical scope and a nose cone was attached. Local reflexes were blocked by applying 1 drop of proparacaine (or similar topical anesthetic) to each eye. ˜1-2 min after proparacaine application the depth of anesthesia was confirmed by gently touching the conjunctiva. Next, the mouse's head was tilted such that the targeted eye is facing the scope. A cotton tipped applicator was used to wipe away any excess liquid. #5 forceps were slid between the upper and lower eyelids such that the eyeball protrudes out of the socket and the conjunctiva was lightly pinch ˜1 mm below the limbus. While holding the eyeball steady with forceps a preloaded 5 μl Hamilton syringe was inserted into the eye at a 60° angle with respect to the optic nerve at ˜1 mm (bevel facing towards the lens) through the sclera ˜1 mm below the limbus into the vitreous cavity.

Purified in vivo grade AAV5.gfaABC₁D with a titer≥1×10¹² vp/ml (viral particles/milliliter) is preferred to efficiently and specifically transduce optic nerve head (ONH) astrocytes. 2 μl of viral particles was then injected. After injecting, the needle was left in the vitreous cavity for ˜15-20 sec to prevent leakage and then was slowly removed. (If liquid starts to flow back out of the site of injection too much virus was injected and the eye should be excluded from future analysis.) Forceps were removed and the eye was gently placed back into the socket by closing the eyelids. A generous amount of Polycin® Ophthalmic Ointment (or similar antibiotic ointment) was applied over the cornea and injection area. The mouse was flipped and the injection was repeated in the contralateral eye. Animals were placed on a heating pad to fully recover (˜2-3 min) before transferring back to their home cages. Expression in ONH astrocytes was detected seven days after injection.

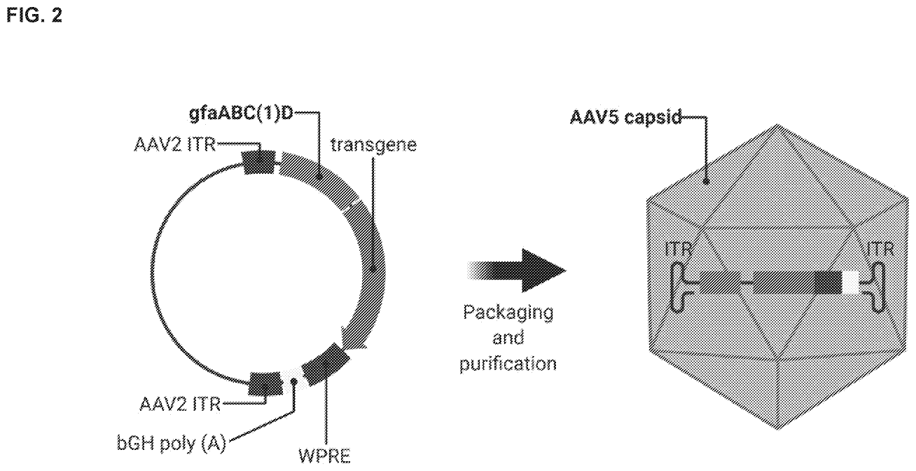

In the present invention, targeted transgene expression in ONH astrocytes is endowed through the combination of two critical elements: A. an AAV packaging vector including the gfaABC₁D promoter and B. an AAV5 viral capsid, as shown in FIG. 2. The packaging vector includes the gfaABC₁D promoter upstream of a transgene coding sequence (which could be recombinant DNA, shRNA, Crisper/Cas9, etc.) followed by a woodchuck posttranscriptional regulatory element (WPRE), and a bovine growth hormone polyadenylation sequence (bGH poly(A)). WPRE increases viral gene expression in packaging cells necessary for production of the high titers required for this invention, and a bGH poly (A) ensures faithful, robust transgene expression in transduced cells. These elements are flanked by two AAV2 inverted terminal repeats (ITR) required for AAV viral packaging. AAV5.gfaABC₁D viral particles should be generated using the herein described packaging vector following standard AAV viral production protocols with an AAV5 rep-cap, and helper plasmids. The virus can be concentrated by polyethylene glycol (PEG) precipitation and further concentrated and purified by cesium chloride (CsCl) gradient ultracentrifugation to produce in vivo grade AAV5.gfaABC₁D virus at titers necessary to efficiently transduce ONH astrocytes (≥1×10¹² viral particle/mL).

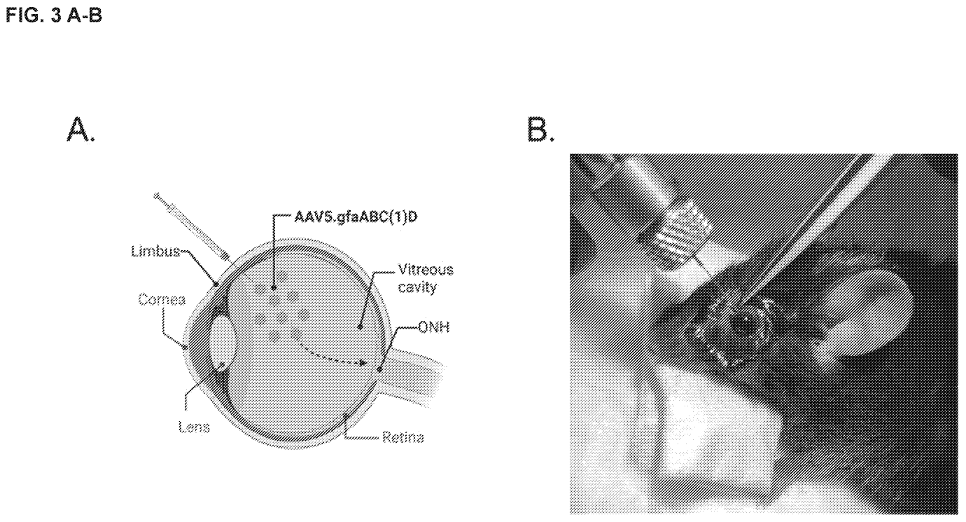

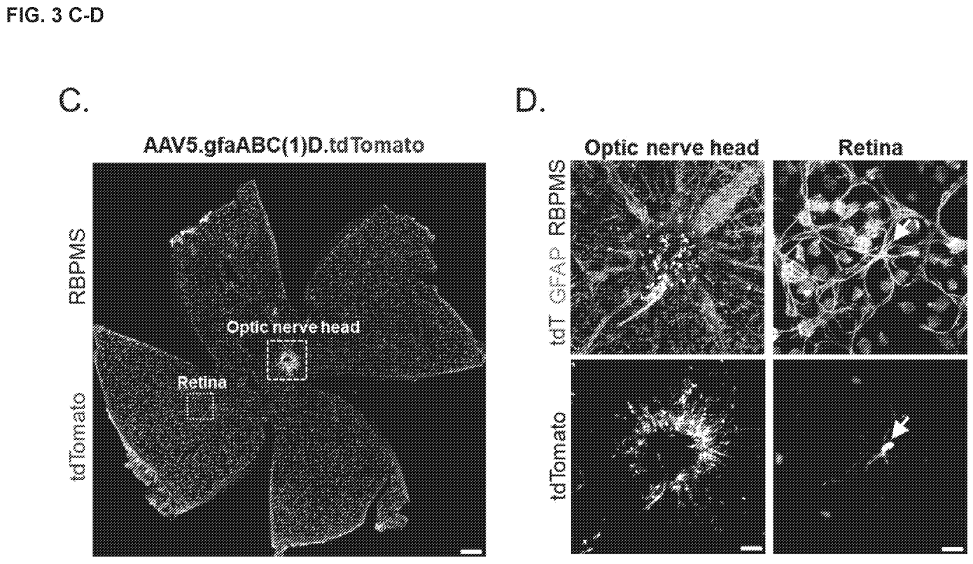

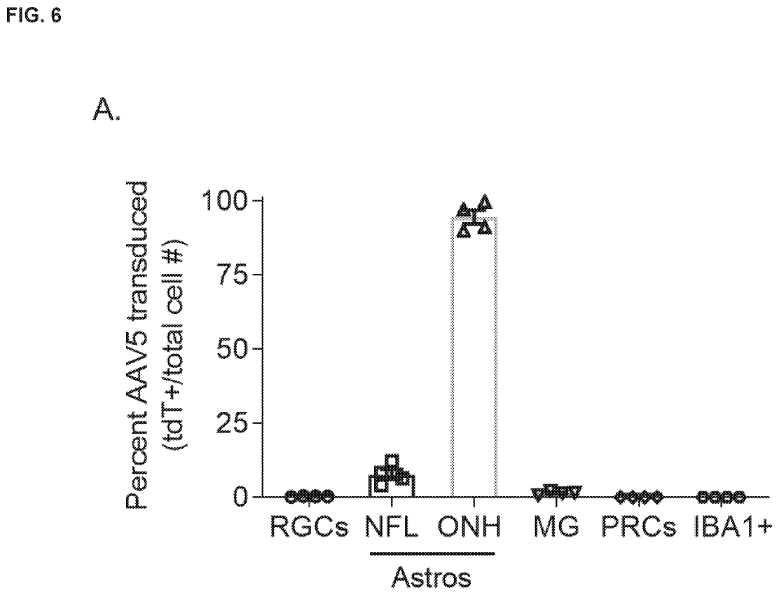

Shown in FIG. 3. intravitreal injection of 2 μls of 1×10⁹ vps/mL of AAV5.gfaABC₁D transduces optic nerve head astrocytes in mice. Virus injection may be accomplished using a preloaded Hamilton syringe inserted at a 60° angle ˜1 mm into the vitreous cavity just below the limbus, as commonly understood by one of ordinary skill in the art. Retinal flat-mount showing AAV5.gfaABC₁D-tdTomato preferentially transduces optic nerve head astrocytes (dashed box in C, magnified in D) and a small subset of nerve fiber layer (NFL, solid arrow) astrocytes identified by GFAP staining (dotted box in C, magnified in D) but not retinal ganglion cells (RGCs) stained with RNA-binding protein with multiple splice (RBPMS). RGC transduction (tdTomato+/RBPMS+) from flat mounts quantified in FIG. 6. Scale in C is 500 μm. Scale in D is 20 μm.

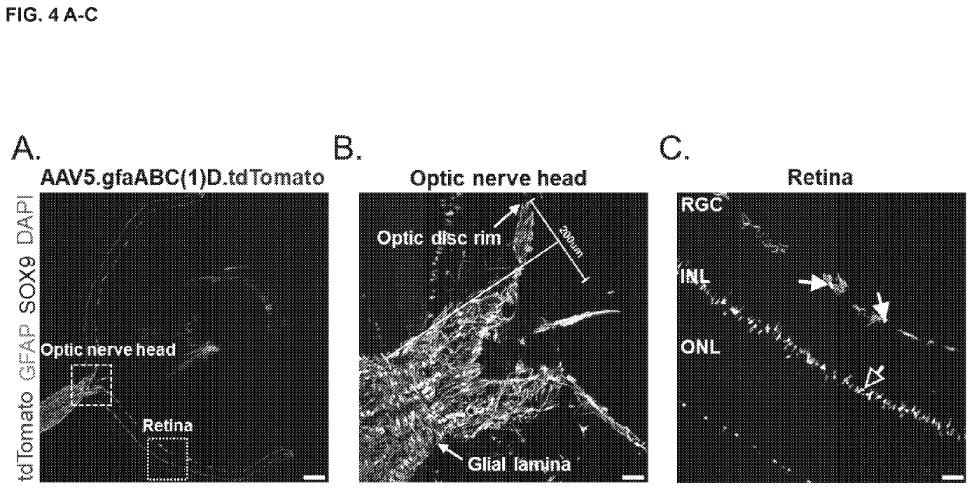

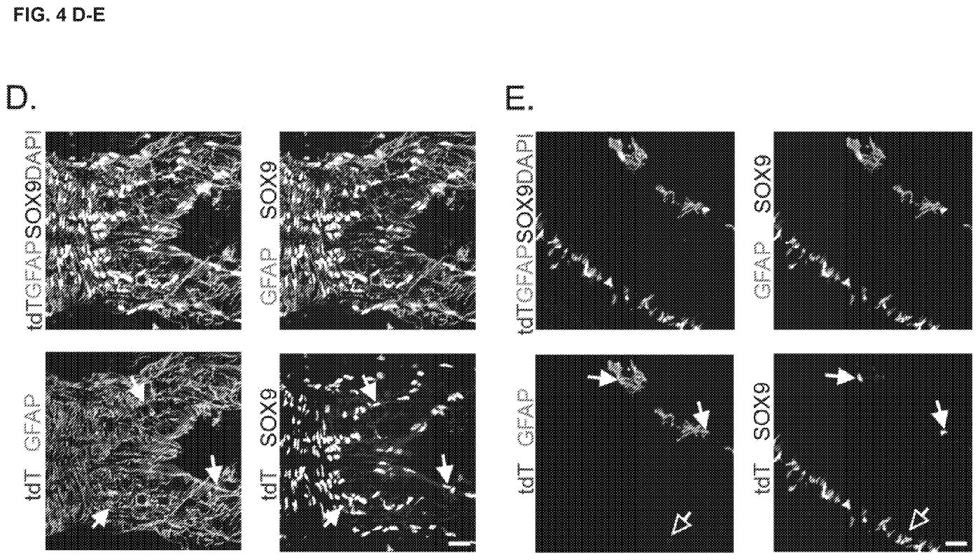

Shown in FIG. 4, intravitreal injection of AAV5.gfaABC₁D preferentially transduces optic nerve head (ONH) astrocytes in mice. A. Cross section of an AAV5.gfaABC₁D-tdTomato injected retina stained with GFAP, Sox9, and DAPI showing high tdTomato expression in the ONH region. Scale is 200 μm. B. ONH region (magnified from dashed box from A) defined vertically by the area between the glial lamina and the surface of the retina and horizontally from the center of the optic disc 200 μm to the optic disc rim. Scale is 50 μm. C. Central retina (magnified from dotted box from A) showing a small subset of nerve fiber layer (NFL, solid arrow) astrocytes as well as Mueller glial (MG, open arrow) are transduced by AAV5.gfaABC₁D-tdTomato. Scale is 50 μm. D. GFAP and Sox9 staining showing high astrocyte transduction (solid arrows) in the defined ONH region between the glial lamina and optic disc rim (magnified from B). E. NFL astrocytes (solid arrows) and Muller glia (MG) in the inner nuclear layer (INL) (open arrow) showing low AAV5. gfaABC₁D-tdTomato expression (magnified from C). Photoreceptors in the outer nuclear layer (ONL) are not transduced (C and E). Scale is 25 μm. Quantification of AAV5.gfaABC₁D.tdTomato transduction efficiencies shown in FIG. 6.

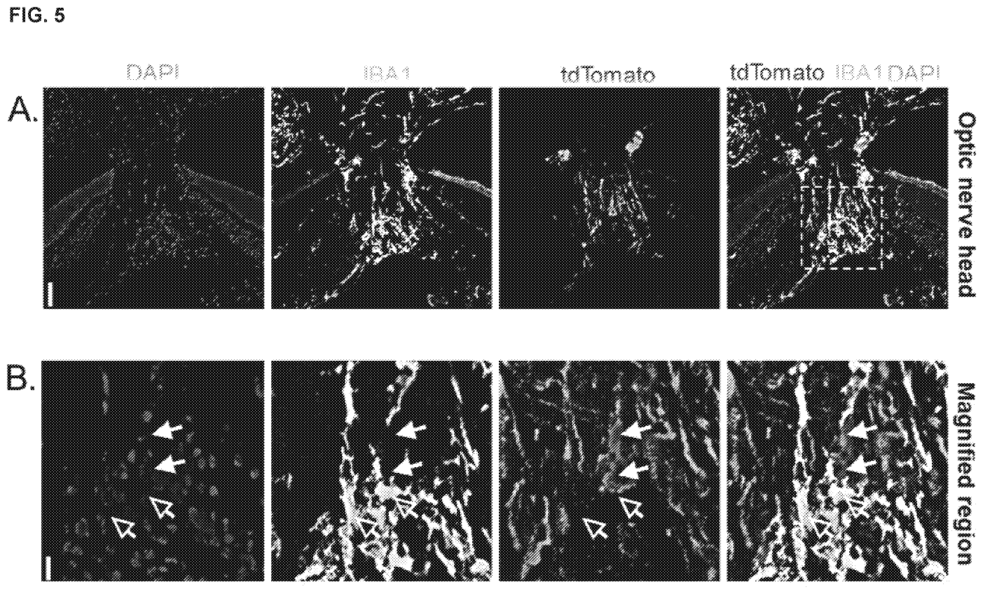

As shown in FIG. 5, AAV5-gfaABC₁D does not transduce microglia in the mouse retina. A. Representative images of IBA1+ stained optic nerve heads showing microglia are not transduced following intravitreal injection of AAV5.gfaABC₁D-tdTomato. Scale is 100 μm. B. Magnified ONH region (dashed box from A) shows IBA+ cells (green; open arrows) do not colocalize with tdTomato (solid arrows; red). Scale is 20 μm. IBA1+ cell transduction (tdTomato+/IBA1+) quantified in FIG. 6.

Intravitreal injection of 2 μls of 1×10⁹ vps/mL of AAV5.gfaABC₁D preferentially transduces optic nerve head (ONH) astrocytes (˜95%), a small subset of nerve fiber (NFL) astrocytes (˜7.5%), and less than 2.5% of muller glia (MG). AAV5.gfaABC₁D does not transduce retinal ganglion cells (RGCs), photoreceptors (PRCs), microglial (IBA+), or any other cell type in the retina. RGC transduction quantified in flat mounts retinas stained with RBPMS (FIG. 3C-D). ONH and NFL astrocytes defined by GFAP and Sox9 expression in the ONH region and retina, respectively, Muller glia (MG) defined by Sox9 and DAPI in the inner nuclear layer (INL), photoreceptors defined by DAPI in the outer nuclear layer (ONL) (FIG. 4D-E), and microglia defined by IBA1 expression (FIG. 5) and quantified in retinal cross sections. Data shown in FIG. 6.

1. A method of genetically modifying retinal astrocytes, the method comprising:
contacting a retinal astrocyte in an eye of a subject with an effective dose of a nucleic acid composition comprising:
a first viral inverted terminal repeat sequence,
a promoter,
a transgene,
a posttranslational regulatory element,
a polyadenylation sequence and
a second viral inverted terminal repeat sequence;
wherein the nucleic acid sequence is encapsulated by a viral capsid to form a viral particle.

2. The method of claim 1, wherein the first viral inverted terminal repeat sequence is AAV2.

3. The method of claim 1, wherein the promoter is gfaABC₁D (SEQ ID NO:1) or a variant thereof.

4. The method of claim 1, wherein the posttranslational regulatory element is a woodchuck posttranslational regulatory element.

5. The method of claim 1, wherein the polyadenylation sequence is a bovine growth hormone polyadenylation sequence.

6. The method of claim 1, wherein the second viral inverted terminal repeat sequence is AAV2.

7. The method of claim 1, wherein the first viral inverted terminal repeat sequence is the same as the second viral inverted terminal repeat.

8. The method of claim 1, wherein the viral capsid is AAV5.

9. The method of claim 1, wherein the effective dose comprises at least 1×10⁸ viral particles per ml.

10. The method of claim 1, wherein the composition is administered via intravitreal injection.

11. The method of claim 1, wherein the retinal astrocyte is an optic nerve head astrocyte.

12. The method of claim 1, wherein the composition comprises ultra-purified viral particles having <0.01% contamination of the total composition.

13. The method of claim 1, wherein the transgene comprises a CRISPR/Cas system.

14. The method of claim 1, wherein the composition does not genetically modify microglia, retinal ganglion cells (RGCs) or photoreceptors (PRCs).

15. The method of claim 1, wherein the subject has or is predicted to have an optic neuropathy.

16. The method of claim 15, wherein the optic neuropathy is pre-glaucoma, glaucoma, ischemic optic neuropathy, or diabetic retinopathy.


SEQUENCE LISTING (1 sequence)

SEQ ID NO: 1 - DNA, 681 bp, Artificial Sequence
  synthetic sequence
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc